(19) United States
(12) Patent Application Publication
(10) Pub. No.: US 2017/0142784 A1
(43) Pub. Date: May 18, 2017

(54) DEVICE FOR HEATING LIQUIDS THAT ARE TO BE DELIVERED TO THE HUMAN BODY

(71) Applicant: Olympus Winter & Ibe GmbH, Hamburg (DE)

(72) Inventor: Michael WOLTER, Hamburg (DE)

(73) Assignee: OLYMPUS WINTER & IBE GMBH, Hamburg (DE)

(21) Appl. No.: 15/129,981

(22) PCT Filed: Feb. 10, 2015
(86) PCT No.: PCT/EP2015/052729

§ 371 Date: Sep. 28, 2016

(30) Foreign Application Priority Data
Mar. 28, 2014 (DE) 10 2014 104 396.0

(51) Int. Cl. H05B 6/64; A61M 3/02; H05B 6/80

(52) U.S. Cl. CPC H05B 6/647; H05B 6/802; H05B 6/6452; A61M 3/0254; A61M 2205/3653; A61M 2205/3686; A61M 2205/3368

(57) ABSTRACT

A device for heating liquids to be supplied to the human body includes an electric heating, a conduit for passing liquid there through, which conduit includes a conduit body on which the electric heating acts for supplying heat, and a heating which acts by electromagnet radiation on the liquid in order to heat it, characterized in that the heating is a microwave heating which includes a microwave generator and a housing which is shielded against microwaves and which includes a cavity in which the microwave generator generate a microwave field, and that a section of the conduit is disposed to extend in the cavity.

The present invention is directed to a device for heating liquids that are to be delivered to the human body, said device comprising a first electric heating plate, a conduit for the passage of the liquid, which conduit comprises a conduit body on which the first electrical heating acts, and a second heating which subjects the liquid to electromagnetic radiation in order to heat the liquid.

The invention is in particular directed to devices for heating irrigation fluids, for example for rinsing with physiological saline solution during laparotomies, pelviscopies, and laparoscopies. Such devices are utilized during minimally invasive surgeries and other surgical procedures to clean the area of the operation by rinsing. Rinsing fluid mixed with blood, tissue residues and other particles is then extracted again using a further device. Main application areas are in urology (in particular prostate resections) and gynecology (in particular endometrial resections). Furthermore, such devices are utilized to perform abdominal or pelvic rinses which may be required during or after a pelviscopy or a laparoscopy. Irrigation liquids are typically provided in plastic bags. A tube is connected to the bottom of the bag. In addition a pressure control unit and a pump are provided such that the irrigation liquid can be conveyed through a tube to the site of application at a desired pressure.

Not all irrigation devices utilize devices for heating the irrigation liquid. However, it turned out that use of irrigation liquids that are not pre-heated or which are not precisely pre-heated to body temperature involves considerable disadvantages and risks. When using a precisely pre-heated irrigation liquid with 36.6° C. the risk of infection is about 6%, but the risk of infection increases to 19% when using a not sufficiently pre-heated irrigation liquid with a temperature of 34.7° C. Furthermore, adverse effects on the cardio vascular system and the nervous system have been observed in connection with insufficiently pre-heated irrigation liquids. In addition, insufficiently pre-heated irrigation liquids lead to a locally lowered body temperature at the application site which leads to a deterioration of blood coagulation in this region. Furthermore, at has been found that when using insufficiently pre-heated irrigation fluids the period of hospitalization on average is 2.6 days longer. Finally, a cool irrigation liquid causes bleedings to recede, which bleedings are then not immediately apparent and which may then, because of lack of treatment, later cause complications.

Conventional devices for heating of irrigation fluids utilize electric heaters, for example heating plates comprising a planar heating surface, wherein the heating plate is brought into surface contact with the plastic bag containing the irrigation liquid. In this connection significant heat losses occur on the path from the heat source to the liquid since several interfaces and materials have to be passed. Also, the contact surface is often not exactly reproducible or changes in the course of time during emptying the plastic bag, for example when the plastic bag containing the irrigation liquid rests on a heating plate as described in EP 0 800 835 A2.

In addition, conventional devices which employ heating plates or counter-flow heat exchangers for heating the irrigation liquid need relatively long heating times. Closely linked to this is a further disadvantage, namely a relatively long response time in which the heating device can be adjusted to a changed heating demand resulting from changed flow or changed input temperature of the irrigation liquids. In other words, conventional heating devices are rather inert and are not capable of quickly adjusting to abrupt changes of operation conditions (changes in volume flow or changes in input temperatures of the irrigation liquids).

There are also devices which employ a conventional heat lamp to heat the irrigation liquid. Such devices have due to their design a very low efficiency since on the one hand only a small portion of the energy input is converted to the desired infrared radiation, and since on the other hand only a small fraction of the infrared radiation generated by the infrared heat lamp reaches the irrigation liquid and is absorbed by this.

A device according to the preamble of claim 1 is known from U.S. Pat. No. 7,153,285 B2 which is in particular directed to a device for heating dialyses liquid. The device comprises a housing which is partitioned into an inbound conduit space and an outbound conduit space. The outbound conduit space has a transparent outer wall onto which a perpendicularly oriented infrared radiation source is directed. On the opposite wall of the outbound conduit space a reflector is attached on the inside. In this manner the infrared radiation emitted by the infrared radiation source is passing through the outbound conduit space perpendicularly to its longitudinal direction and to the flow direction of the liquid, and is reflected at the reflector and finally exits through the transparent outer wall again. When passing through the liquid a fraction of the infrared radiation is absorbed which results in heating of the through-flowing liquid. In addition, an electrical heating plate is disposed on the opposite side of the infrared radiation source in heat conducting contact. The electric heating plate, which is only slowly adjustable with respect to its thermal output, is intended to take over the basic load of the heating demand, whereas the infrared radiation source, which is quickly adjustable in its heating power, is intended to absorb sudden demand peaks for heating power.

This construction is relatively complicated since it requires a housing with a separate, transparent outer wall and with a reflector attached to the opposite wall in the interior of the conduit. Furthermore, the volume flow of irrigation liquids is usually higher than that of liquids to be infused, such as dialyses liquids. On the other hand, since the heat output of typical available IR radiation sources, such as IR-LEDs, is rather limited the heating device is not capable of reacting to rapid demand changes with sufficient heat output, for example to a rapid increase in heat output demand following a rapid increase of the volume flow of the liquid.

Therefore, it is an object of the present invention to provide a device for heating liquids to be delivered to a human body, which device is of simple construction and inexpensive to manufacture, and which device is capable effectively heating also larger volume flows to body temperature, and which device is quickly adjustable to changed demands on the heating power, also in cases of larger power changes.

This object is achieved by a device comprising the features of claim 1. Preferred embodiments of the invention are set out in the dependent claims.

According to the present invention a second heating is provided besides a first electric heating in heat conducting contact with a conduit body, which second heating is acting by electromagnet radiation and is formed as a microwave heating. The microwave heating comprises a microwave generator and a housing which is shielded against microwaves and encloses a cavity in which a microwave field is generated by the microwave generator. A section of the conduit is disposed to extend through the cavity of the housing such that through-flowing liquid is exposed to the microwave field in the cavity of the housing. The section of the conduit is permeable to microwaves so that through-flowing liquid when passing the section of the conduit through the cavity is heated in the microwave field.

Microwave heatings which are in principle constructed similar to microwave ovens for domestic use are available at relatively low costs. The heat output is adjustable very quickly and over a large dynamic range. Since a very rapidly changed setting of the heat output of the microwave generator immediately results in a correspondingly changed absorption of microwave power, the heating power effectively applied by the microwave heating can be adjusted almost instantaneously.

Preferably, the section of the conduit extending through the housing is, in flow direction of the liquid, disposed downstream of the conduit body in contact with the electrical heating. In this manner the heating power applied by the electrical heating and by the microwave heating is applied to the liquid sequentially in time. Therefore, it is in principle possible to adjust the microwave heating in dependence on the heating achieved by the electric heating.

For this purpose a temperature sensor is disposed on the conduit for measuring the temperature in the interior thereof, wherein the temperature sensor is disposed upstream of the section of the conduit extending through the cavity. Further, a control unit is provided which is arranged to adjust the power of the microwave generator in dependence on the temperature determined by the temperature sensor in such a manner that the liquid during the passage of the conduit section through the cavity is supplied with precisely so much energy that a predetermined target temperature is reached.

In a preferred embodiment it is alternatively possible that downstream of the conduit section extending through the cavity a temperature sensor is provided on the conduit for measuring the temperature of the liquid therein. A control unit is also provided which is arranged to adjust the output power of the microwave generator in dependence on the temperature determined by the temperature sensor downstream of the microwave heating to readjust it such that the liquid after passing the conduit section through the cavity has a predetermined target temperature.

In this embodiment a further temperature sensor may be disposed on the conduit for measuring the temperature of the liquid in the conduit, which further temperature sensor is disposed downstream of the electric heating plate and upstream of the section of the conduit extending through the cavity. In this case the control unit is further arranged to control the power of the electric heating plate in dependence on the temperature determined by the further temperature sensor downstream of the heating plate such that the liquid after passing the conduit body in contact with the electric heating plate is almost at the predetermined target temperature. In this manner the need for additional heating power from the microwave heating can be minimized.

In an advantageous embodiment the section of the conduit extending through the cavity is configured with a meandering course or with at least one winding to thereby increase the length of the flow path effectively available for the microwave heating.

The microwave generator of the microwave heating may comprise a magnetron.

The section of the conduit extending through the cavity can be formed by a plastic tube. The plastic tube may be made of polyvinyl chloride or silicone.

The invention will be described below with reference to an embodiment with reference to the drawings in which:

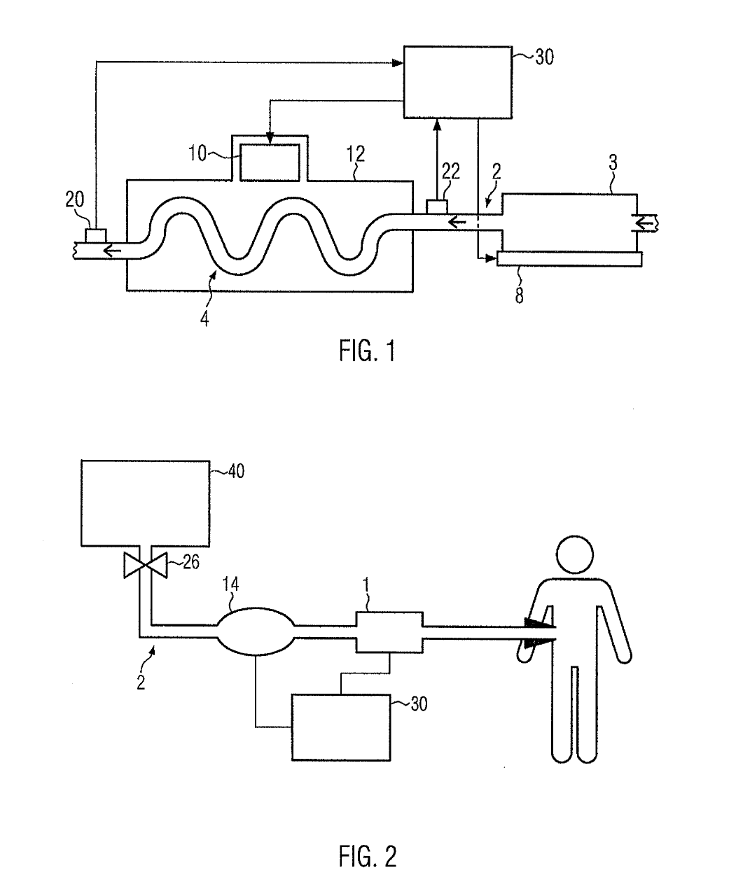

FIG. 1 shows a schematic representation of a device for heating liquids to be supplied to the human body, and

FIG. 2 shows a schematic representation of a device for delivering heated liquids to be supplied to the human body.

The device shown in FIG. 1 comprises a conduit 2 for supplying a liquid to an area of application in a human body, for example for supplying an irrigation liquid to a surgical site. The conduit 2 includes a conduit body 3 which is shown schematically only and which is in heat conducting contact with a first electric heating in the form of an electric heating plate 8. As first electric heating also an electric heating coil can be used with surrounds a section of the conduit. The course of the flow path through the conduit body 3 is not shown in detail. The flow path may be designed for example by meandering flow line paths which creates a substantially increased flow path length in heat conducting contact with the electric heating plate 8.

In the embodiment shown in FIG. 1 the conduit body 3 has an input port. That is not necessarily the case because the conduit body 3 could in principle also be formed by a liquid reservoir, for example by a plastic bag, in which the irrigation liquid is provided.

After leaving the conduit body 3 conduit 2 extends further to a housing shielded against microwaves, which housing encloses a cavity in which a microwave generator 10 generates a microwave field. The conduit 2 includes a section 4 running through the cavity of housing 12 in the microwave field. The section 4 may have, as schematically indicated, a meandering course to increase the effective length of the flow path of the liquid in the microwave field.

On the conduit 2 a temperature sensor 22 is disposed downstream of the conduit body 3, which temperature sensor serves to sense the temperature of the liquid flowing through the conduit and which is connected to a control unit 30. In addition, a temperature sensor 20 is disposed on the conduit 2 downstream of the housing 12 of the microwave heating, which temperature sensor 20 serves to sense the temperature of the liquid in the conduit after passing the microwave heating. The temperature sensor 20 is likewise connected to the control unit 30.

The control unit 30 is arranged to adjust the heating power 8 in dependence on the temperature determined by the temperature sensor 22 in such a manner that the temperature is as close as possible to a predetermined target temperature, but is below this target temperature. The control unit 30 is further arranged, to adjust the heating power of the microwave generator 10 in dependence on the temperature determined by the temperature sensor 22 in such a manner that the heating power applied by the microwave heating brings the liquid to the predetermined target temperature. Alternatively or in addition the control unit 30 may be arranged to regulate the heating power of the microwave generator 10 in addition in dependence on the temperature determined by temperature sensor 20 in such a manner that the target temperature is set as accurately as possible.

FIG. 2 shows a schematical view of a device for supplying irrigation liquid for rinsing a surgical site. There is a reservoir container 40 for irrigation liquid to which a conduit 2 is connected for conveying irrigation liquid. In the conduit 2 at least one valve 26 is present. Furthermore, a controllable pump 14 is provided which conveys the irrigation liquid through the conduit 2 to the surgical site. The output level of the pump 14 is adjusted by a control unit 30 such that a desired pressure of the irrigation liquid is generated. Downstream of the pump 14 a device 1 for heating the irrigation liquid to be supplied to a human body is generally shown. This device includes a microwave heating as described in the present specification and may have the construction as shown in FIG. 1. The heating power of the microwave heating is controlled by the control unit 30.

1. Device for heating liquids to be supplied to the human body, said device comprising a first electric heating, a conduit for passing liquid there through, which conduit includes a conduit body on which the first electric heating acts to supply heat, and a second heating which acts by electromagnetic radiation on the liquid to heat it, wherein the second heating is a microwave heating which comprises a microwave generator and a housing shielded against microwaves and including a cavity in which the microwave generator generates a microwave field, and in that a section of the conduit is disposed to extend in the cavity.

2. Device according to claim 1, wherein the section of the conduit extending in the cavity is disposed, in flow direction of the liquid, downstream of the conduit body which is in heat conducting contact with the first electric heating.

3. Device according to claim 2, wherein upstream of the section of the conduit extending in the cavity a temperature sensor is disposed on the conduit for measuring the temperature of the liquid therein, and in that a control unit is provided which is arranged to adjust the power of the microwave generator in dependence on the temperature determined by the temperature sensor in such a manner that such an amount of energy is supplied to the liquid while passing the conduit section through the cavity so that a predetermined target temperature is reached.

4. Device according to claim 2, wherein downstream of the section of the conduit extending in the cavity a temperature sensor is disposed on the conduit for measuring the temperature of the liquid therein, and in that a control unit is provided which is arranged to regulate the power of the microwave generator in dependence on the temperature determined by the temperature sensor in such a manner that the liquid after passing the conduit section through the cavity reaches a predetermined target temperature.

5. Device according to claim 4, wherein downstream of the first electric heating and upstream of the section of the conduit extending in the cavity a further temperature sensor is disposed on the conduit for measuring the temperature of the liquid therein, and in that the control unit is further arranged, to regulate the power of the electric heating in dependence on the temperature determined by the further temperature sensor in such a manner that the liquid after passing the conduit body in contact with the electric heating is as close as possible below the predetermined target temperature.

6. Device according to claim 1, wherein the section of the conduit extending through the cavity has a meandering course or is provided with at least one winding.

7. Device according to claim 1, wherein the microwave generator comprises a magnetron.

8. Device according to claim 2, wherein the section of the conduit extending through the cavity has a meandering course or is provided with at least one winding.

9. Device according to claim 3, wherein the section of the conduit extending through the cavity has a meandering course or is provided with at least one winding.

10. Device according to claim 4, wherein the section of the conduit extending through the cavity has a meandering course or is provided with at least one winding.

11. Device according to claim 5, wherein the section of the conduit extending through the cavity has a meandering course or is provided with at least one winding.

12. Device according to claim 2, wherein the microwave generator comprises a magnetron.

13. Device according to claim 3, wherein the microwave generator comprises a magnetron.

14. Device according to claim 4, wherein the microwave generator comprises a magnetron.

15. Device according to claim 5, wherein the microwave generator comprises a magnetron.

16. Device according to claim 6, wherein the microwave generator comprises a magnetron.

17. Device according to claim 8, wherein the microwave generator comprises a magnetron.

18. Device according to claim 9, wherein the microwave generator comprises a magnetron.

19. Device according to claim 10, wherein the microwave generator comprises a magnetron.

20. Device according to claim 11, wherein the microwave generator comprises a magnetron.
